(19) United States
(12) Patent Application Publication
(10) Pub. No.: US 2026/0151639 A1
(43) Pub. Date: Jun. 4, 2026

(54) Treatment Device

(71) Applicants: SUMIDA CORPORATION, Tokyo (JP); National University Corporation, Iwate University, Morioka-shi (JP); ITO CO., LTD., Tokyo (JP)

(72) Inventors: Kenji TERAO, Natori City (JP); Masaki SAITO, Natori City (JP); Masahiro DAIBO, Morioka (JP); Keita TAKANAKA, Bunkyo-ku (JP); Tatsuya NAKAMURA, Bunkyo-ku (JP)

(21) Appl. No.: 19/460,435

(22) Filed: Jan. 27, 2026

Related U.S. Application Data
(63) Continuation of application No. PCT/JP2024/022770, filed on Jun. 24, 2024.

(30) Foreign Application Priority Data
Aug. 10, 2023 (JP) 2023-130712

(51) Int. Cl. A61N 1/40; A61N 7/00; A61N 7/02

(52) U.S. Cl. CPC A61N 1/40; A61N 7/02; A61N 2007/0004

(57) ABSTRACT

A vector potential coil device generates a vector potential and applies electrical stimulation to an affected area using the generated vector potential. A heating means (actuator) heats the affected area. A vector potential coil drive device drives the vector potential coil device. A high frequency power supply drives the actuator. A controller controls the vector potential coil drive device and the high frequency power supply, causes the vector potential coil device to generate the vector potential, and causes the heating means to heat the affected area under a specified condition based on the combined therapy of thermotherapy and electrical stimulation.

### CROSS REFERENCE TO RELATED APPLICATIONS

This application is a continuation of PCT Application No. PCT/JP2024/022770, filed on Jun. 24, 2024, which claims priority to Japanese Patent Application No. 2023-130712, filed on Aug. 10, 2023. The contents of both of the above applications are expressly incorporated herein by reference in their entirety.

### BACKGROUND

### Technical Field

The present invention relates to a treatment (therapy) device.

### Related Art

For instance, a treatment combining thermotherapy and electrical (electric) stimulation has been proposed as a treatment method for non-alcoholic fatty liver disease (NAFLD) (for instance, refer to a Non-Patent Literature, “Activation of heat shock response improves biomarkers of NAFLD in patients with metabolic diseases”, Kondo et al., Endocrine Connections (European Journal of Endocrinology), 2021). In the above-mentioned treatment method, an electric current (electricity is conducted) and heat are applied by a pair of pads for an electric thermal treatment device. At that time, one pad is attached to the back and the other is attached to the abdomen, and electricity is conducted between the pads. At the same time, the pads themselves are heated to 42 degrees Celsius. It has been reported that applying electrical stimulation in the above warmed state enhances the therapeutic effect.

On the other hand, a treatment device with an applicator has been proposed. The applicator stably supplies physical energy to an affected area using an ultrashort wave, a microwave, an ultrasonic wave (for instance, refer to Japanese Patent Publication Number 2020-62154).

Further, in recent years, a vector potential generation device has been developed (for instance, refer to International Patent Publication Number WO2015/099147 and Japanese Patent Number 6205572). The vector potential generation device generates a vector potential by conducting a current through a vector potential coil that is formed by circulating a solenoid coil.

In the above-mentioned treatment method, two pads are attached to the back and the abdomen and electricity is conducted through the surface of the human body so as to provide electrical stimulation. As a result, it imposes a great burden on a patient.

### SUMMARY

The present invention has been made in view of the above issue. The present invention has an object that is to obtain a treatment device that imposes less burden on a patient when performing a treatment that combines thermotherapy and electrical stimulation.

A treatment device according to the present invention includes a vector potential coil device that generates a vector potential and applies electrical stimulation to an affected area using the generated vector potential, a heating means that heats the affected area, a vector potential coil driver (drive device) that drives the vector potential coil device, a high frequency power supply that drives the heating means, and a controller that controls the vector potential coil driver and the high-frequency power supply, and causes the vector potential coil device to generate the vector potential and the heating means to heat the affected area under a specified condition based on a combined therapy of hyperthermia and electrical stimulation.

### Effects of the Invention

According to the present invention, it is possible to obtain a treatment device that imposes less burden on a patient when performing a treatment that combines thermotherapy and electrical stimulation.

### BRIEF DESCRIPTION OF THE DRAWINGS

FIG. 1 is a block diagram that shows a configuration of a treatment device according to an embodiment of the present invention.

FIG. 2 is a diagram that shows an example of a vector potential coil with respect to a vector potential coil device 1 shown in FIG. 1.

FIG. 3 is a block diagram that shows a configuration example of a vector potential coil drive device 2 shown in FIG. 1.

FIG. 4 is a block diagram that shows a configuration example of a high frequency power source 4 shown in FIG. 1.

FIG. 5 is a diagram that shows a configuration of a vector potential coil device 1 according to a second embodiment of the present invention.

FIG. 6 is a diagram that shows a configuration of a vector potential coil device 1 with respect to a treatment device according to a third embodiment of the present invention.

FIG. 7 is a diagram that shows a configuration of a vector potential coil device 1 with respect to a treatment device according to a fourth embodiment of the present invention.

FIG. 8 is a front view that shows an example of a vector potential coil according to a fifth embodiment of the present invention.

FIG. 9 is a top view that shows an example of the vector potential coil according to the fifth embodiment of the present invention.

FIG. 10 is a side view that shows an example of the vector potential coil according to the fifth embodiment of the present invention.

FIG. 11 is a side view that shows a treatment device according to a sixth embodiment of the present invention.

FIG. 12 is a diagram that shows an example of a vector potential coil device 1 according to a seventh embodiment.

FIG. 13 is a diagram that shows a configuration of a vector potential coil device 1 with respect to a treatment device according to an eighth embodiment of the present invention.

FIG. 14 is a diagram that explains an arrangement of a vector potential coil device 1 and an actuator 3 relative to a human body according to a ninth embodiment.

FIG. 15 is a diagram that explains an arrangement of a vector potential coil device 1 and an actuator 3 relative to a human body according to a tenth embodiment.

FIG. 16 is a diagram that explains an arrangement of a vector potential coil device 1 and an actuator 3 relative to a human body according to an eleventh embodiment.

FIG. 17 is a diagram that explains an arrangement of a vector potential coil device 1 and an actuator 3 relative to a human body according to a twelfth embodiment.

FIG. 18 is a diagram that explains an arrangement of a vector potential coil device 1 and an actuator 3 relative to a human body according to a thirteenth embodiment.

FIG. 19 is a diagram (1/3) that shows examples of drive waveforms of a vector potential coil device 1 and an actuator 3 with respect to a treatment device according to a fourteenth embodiment.

FIG. 20 is a diagram (2/3) that shows examples of drive waveforms of the vector potential coil device 1 and the actuator 3 with respect to the treatment device according to the fourteenth embodiment.

FIG. 21 is a diagram (3/3) that shows examples of drive waveforms of the vector potential coil device 1 and the actuator 3 with respect to the treatment device according to the fourteenth embodiment.

FIG. 22 is a perspective view that shows a cylindrical-shaped (tube-shaped) applicator 10. The cylindrical-shaped (tube-shaped) applicator 10 is configured with an actuator 3 and a cylindrical-shaped (tube-shaped) vector potential coil device 1. The cylindrical-shaped (tube-shaped) vector potential coil device 1 is configured with a plurality of VP coils 11. An arm of a patient is inserted into an inner side of the cylindrical-shaped (tube-shaped) vector potential coil device 1.

FIG. 23 is a perspective view that shows a belt-shaped applicator 10. The belt-shaped applicator 10 is configured with a coil 3a in a planar coil shape and a belt-shaped vector potential coil device 1 in which a VP coil 11 or a plurality of VP coils are housed.

FIG. 24 is a block diagram that shows a configuration of a treatment device according to a fifteenth embodiment of the present invention.

### DETAILED DESCRIPTION

Embodiments of the present invention will be explained below with reference to the drawings.

### First Embodiment

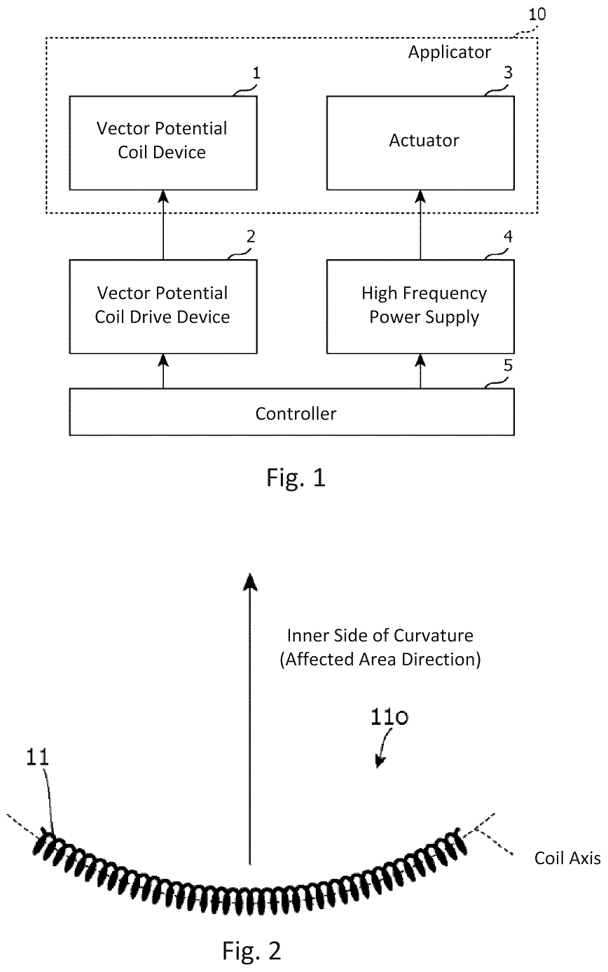

FIG. 1 is a block diagram that shows a configuration of a treatment device according to an embodiment of the present invention. The treatment device shown in FIG. 1 is a treatment device for a combined therapy of thermotherapy and electrical stimulation. The treatment device has a vector potential coil device 1, a vector potential coil drive device (driver) 2 for the vector potential coil device 1, an actuator 3, a high frequency power source 4 for the actuator 3, and a controller 5 that controls the vector potential coil drive device 2 and the high frequency power source 4.

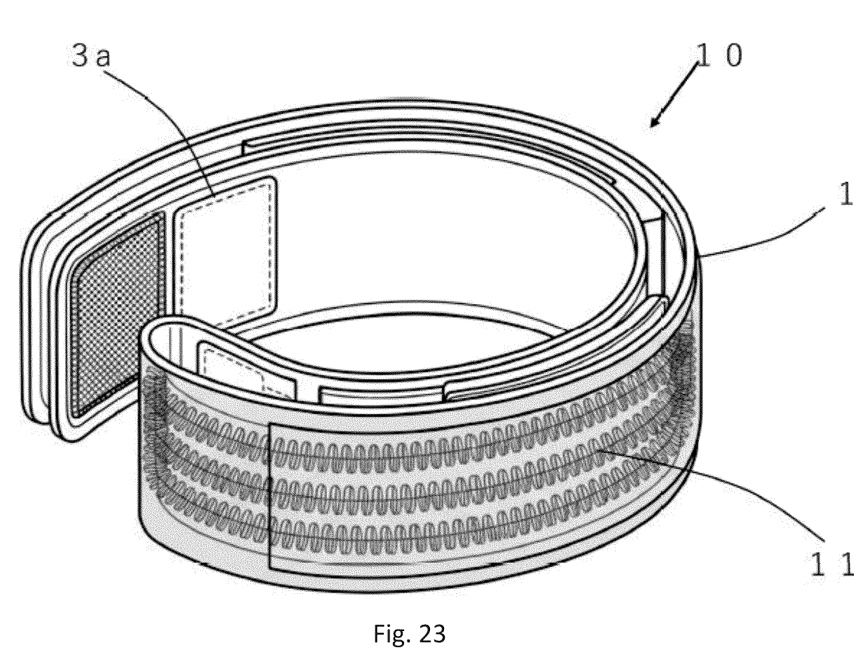

The vector potential coil device 1 generates a vector potential and applies an electrical stimulation to an affected area using the generated vector potential. The vector potential coil device 1 includes one or more solenoid coils arranged along a predetermined planar shape (see FIG. 23) or a predetermined curved surface shape, and a vector potential is generated by the one or more solenoid coils.

FIG. 2 is a diagram that shows an example of a vector potential coil with respect to the vector potential coil device 1 shown in FIG. 1. The vector potential coil device 1 has, for instance, the vector potential coil (also referred to as a “VP coil” below) 11 as shown in FIG. 2. The VP coil 11 is a solenoid coil extending along a curved coil axis.

The coil axis of the VP coil 11 does not go around once (one revolution) or more. For instance, the above-mentioned coil axis is in a circular arc shape. Further, an angle (central angle) from one end to the other end of the VP coil 11 (the coil axis of the VP coil) when viewed from a center of a circle including the coil axis (i.e., the circular arc) is less than 360 degrees. As a result, the opening 11o is formed. For instance, the central angle may be 180 degrees or may be less than 180 degrees. However, the larger the central angle is, the greater the intensity of the vector potential in the inner side of the curvature becomes. Thus, it is preferred that the central angle is large. The central angle is any angle greater than 0 degrees and less than 360 degrees, and may further be (a) any angle greater than 0 degrees and equal to or less than 180 degrees, (b) any angle greater than 0 degrees and equal to or less than 90 degrees, (c) any angle greater than 0 degrees and equal to or less than 45 degrees, or (d) any angle equal to or greater than 0.5 degrees and less than 360 degrees, and further, (e) any angle equal to or greater than 0.5 degrees and equal to or less than 180 degrees, (f) any angle equal to or greater than 0.5 degrees and equal to or less than 90 degrees, (g) any angle equal to or greater than 0.5 degrees and equal to or less than 45 degrees, (h) any angle equal to or greater than 0.5 degrees and equal to or less than 25 degrees, or (i) any angle equal to or greater than 2 degrees and less than 360 degrees, further, (j) any angle equal to or greater than 2 degrees and equal to or less than 180 degrees, (k) any angle equal to or greater than 2 degrees and equal to or less than 90 degrees, (l) any angle equal to or greater than 2 degrees and equal to or less than 45 degrees, (m) any angle equal to or greater than 2 degrees and equal to or less than 25 degrees, or (n) any angle equal to or greater than 5 degrees and less than 360 degrees, further, (o) any angle equal to or greater than 5 degrees and equal to or less than 180 degrees, (p) any angle equal to or greater than 5 degrees and equal to or less than 90 degrees, (q) any angle equal to or greater than 5 degrees and equal to or less than 45 degrees, or (r) any angle equal to or greater than 5 degrees and equal to or less than 25 degrees. Furthermore, when it is considered about attachment and detachment of an application target, to which the vector potential is applied, to and from the VP coil 11 from the inner side of the curvature, it is preferred that the opening 11o is to be large (in other words, a curvature radius of the above-mentioned coil axis and/or the above-mentioned central angle are determined according to the shape and size of the application target).

A vector potential generated by a current that is conducted through the VP coil 11 is weakened as it moves away from the current. However, since the VP coil 11 (the coil axis thereof) is curved as mentioned above, an intensity of the vector potential becomes greater at an inner side (an inner direction) of the curvature (a curvature center in the case of a circular arc shape). Specifically, the vector potential being generated by the current at each position of the VP coil 11 overlaps at the inner side of the curvature.

The vector potential coil drive device 2 shown in FIG. 1 generates a current based on power from, for instance, a commercial power supply or a battery (a primary battery or a secondary battery), and conducts the current (here, an alternating current (AC current) of a predetermined frequency) through the VP coil 11. Here, a waveform of the AC current may be a sine wave, a rectangular wave, a pulse wave, an impulse train (sequence), or a combination of these waves. Further, the AC current may be output steadily (constantly), or may be output the AC current intermittently in bursts that are repeatedly between output and stop.

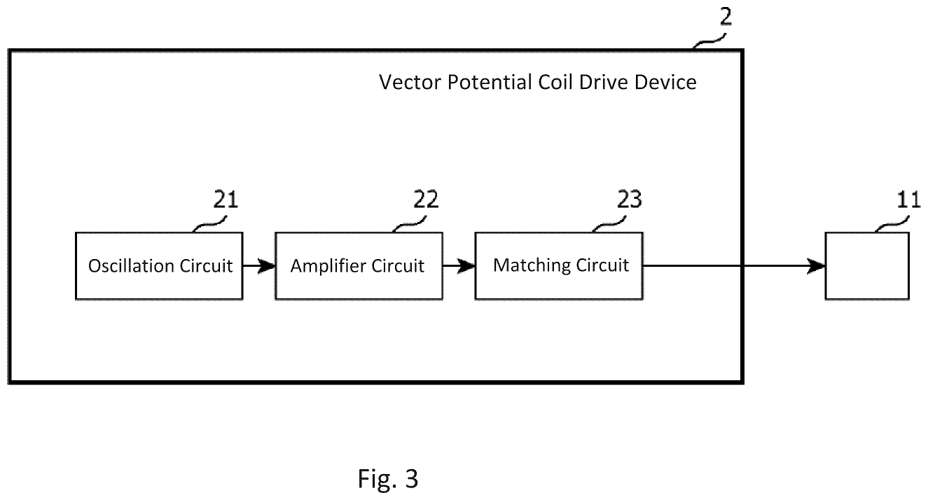

FIG. 3 is a block diagram that shows a configuration example of the vector potential coil drive device 2 shown in FIG. 1. As shown in FIG. 3, the vector potential coil drive device 2 has an oscillator circuit 21, an amplifier circuit 22, and a matching circuit 23. The oscillator circuit 21 generates an AC voltage of a predetermined waveform and frequency. The amplifier circuit 22 performs voltage amplification and power amplification on the AC voltage. Further, the matching circuit 23 performs impedance matching with the VP coil 11. As a result, as mentioned above, a current of a predetermined waveform is conducted through the VP coil 11. An AC vector potential is applied to an affected area. Thus, the electrical stimulation occurs in the affected area.

Refer back to FIG. 1, the actuator 3 generates at least one of an ultrashort wave (ultrashort waves) (very high frequency (VHF)), a microwave (microwaves), and an ultrasonic wave (ultrasonic waves). The actuator 3 may be a coil that generates the ultrashort waves or the microwaves, or a piezoelectric element that generates the ultrasonic waves. The actuator 3 heats the affected area with at least one of the ultrashort waves, the microwaves, and the ultrasonic waves. When the affected area is an internal organ such as a liver, the actuator 3 can directly heat the affected area being deep within a human body using at least one of the ultrashort waves, the microwaves, and the ultrasonic waves. Thus, the actuator 3 can heat the affected area more directly, efficiently, and therefore quickly as compared with a case in which the affected area is heated by heating a pad attached to the human body and then transferring heat from the epidermis of the human body to the affected area. As a result, the treatment time is shortened and the burden on the patient is reduced. The actuator 3 is an example of a heating means for heating the affected area.

The high frequency power source 4 shown in FIG. 1 generates a current based on power from, for instance, a commercial power supply or a battery (a primary battery or a secondary battery), and conducts that current (here, an alternating current (AC current) of a predetermined frequency) through the actuator 3. Here, the waveform of the AC current may be a sine wave, a rectangular wave, a pulse wave, or a combination of these waves. Further, the AC current may be output steadily (constantly), or may be output intermittently in bursts that are repeatedly between output and stop.

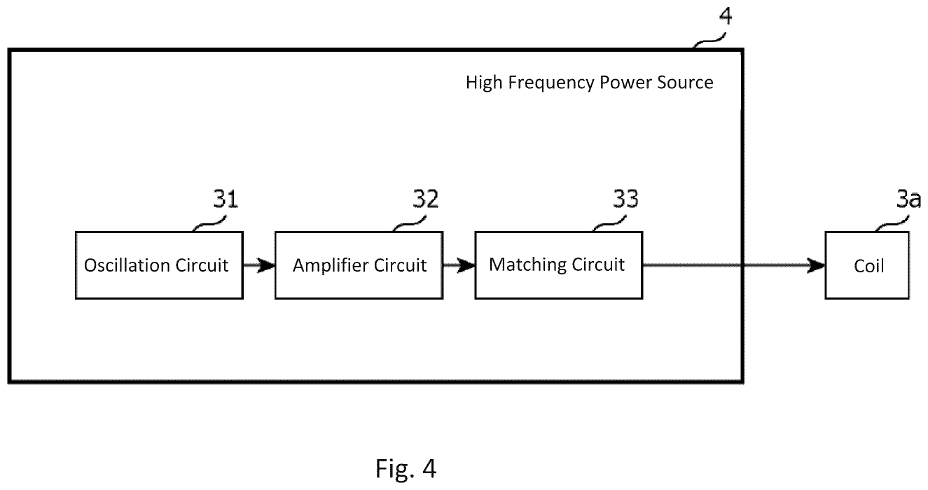

FIG. 4 is a block diagram that shows a configuration example of the high frequency power source 4 shown in FIG. 1. The high frequency power source 4 shown in FIG. 4 is for a case in which the actuator 3 is a coil that generates the ultrashort waves or the microwaves. In this case, as shown in FIG. 4, the high frequency power source 4 has an oscillator circuit 31, an amplifier circuit 32, and a matching circuit 33. The oscillator circuit 31 generates an AC voltage of a predetermined waveform and frequency. The amplifier circuit 32 performs the voltage amplification and the power amplification on the AC voltage. Further, the matching circuit 33 performs impedance matching with a coil 3a of the actuator 3. As a result, a current of a predetermined waveform is conducted through the coil 3a of the actuator 3. Thus, the ultrashort waves or the microwaves are transmitted toward the affected area.

The controller 5 controls the vector potential coil drive device 2 and the high frequency power source 4, and causes the vector potential coil device 1 to generate the vector potential, and at the same time, causes the actuator 3 to generate at least one of the ultrashort waves, the microwaves, and the ultrasonic waves, under specified conditions based on the combined therapy of thermotherapy and electrical stimulation.

For instance, the above-mentioned combined therapy activates a normalization mechanism in biological tissues in the affected area via heat shock proteins or ubiquitinated proteins. For instance, in the case of the above-mentioned combined therapy for non-alcoholic fatty liver disease (NAFLD), the controller 5 causes the vector potential coil device 1 to generate the vector potential, and at the same time, causes the actuator 3 to generate at least one of the ultrashort waves (for instance, 30-300 MHz), the microwaves (for instance, 300 MHz-300 GHz), and the ultrasonic waves (sonic waves not intended for hearing), so as to match the conditions of the electrical stimulation (a pulse current with a pulse shape of 55 Hz or more) that is described in Non-Patent Document 1 above and the thermotherapy (heating at 42 degrees Celsius). For instance, a single treatment (an application of the vector potential and at least one of the ultrashort waves, the microwaves, and the ultrasonic waves) lasts for 10-60 minutes.

Further, in addition to the non-alcoholic fatty liver disease (NAFLD), the combined therapy can also be applied to metabolic diseases such as insulin resistance, hyperglycemia, chronic inflammation, and visceral fat excess. Further, the controller 5 may cause the vector potential coil device 1 to generate the vector potential under the conditions according to those symptoms, and at the same time, may also cause the actuator 3 to generate at least one of the ultrashort waves, the microwaves, and the ultrasonic waves.

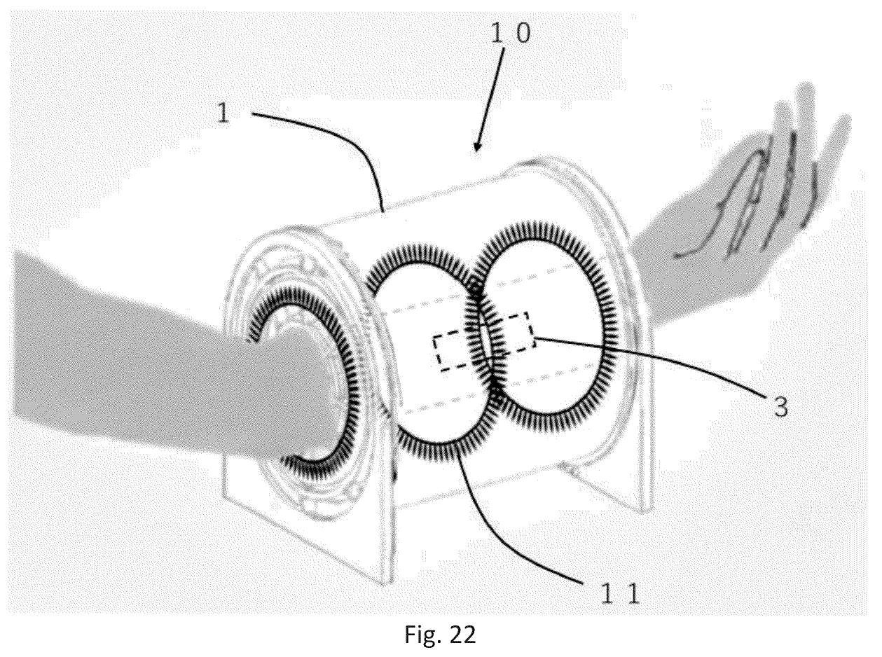

Furthermore, in the first embodiment, the treatment device has a single applicator 10. Further, the vector potential coil device 1 and the actuator 3 are incorporated into the single applicator 10. For instance, the single applicator 10 is not directly attached to the skin of the human body. The single applicator 10 causes the vector potential coil device 1 and the actuator 3 to be arranged at a position in which the vector potential and at least one of the ultrashort waves, the microwaves, and the ultrasonic waves are applied to the affected area. In this case, the actuator 3 may directly come in contact with the skin. The applicator 10 is, for instance, a probe-type applicator or a pad-type applicator. For example, the applicator 10 is configured with a configuration shown in FIG. 22. As shown in FIG. 22, the vector potential coil device 1 is a housing structure that is cylindrical-shaped (tube-shaped). A plurality of VP coils 11 are arranged within the tube-shaped housing structure of the vector potential coil device 1. Further, the actuator 3 is located within the tube-shaped housing structure or on an inner surface of an inner hole, facing a human body, of the tube-shaped housing structure (the vector potential coil device 1). The affected part, such as an arm, of the patient is inserted through the inner hole of the applicator 10. The affected part may not be directly contacted to the VP coils and may be contacted to an inner surface of the tube-shaped housing structure of the vector potential coil device 1. Thus, the hyperthermia treatment (thermotherapy) and electrical stimulation can be performed at the affected part without directly attaching or adhering to a skin of the human body.

Note that, here, the actuator 3 is the coil 3a having a planar coil shape (see FIG. 23). But, the actuator 3 may be of a capacitor type. In the case of the capacitor type, at least two electrodes are used instead of the coil 3a. Further, the electrodes are arranged so as to sandwich the affected area. As a result, the electrical stimulation in a band of the ultrashort waves or the microwaves is applied.

Further, here, the vector potential coil device 1 and the actuator 3 are incorporated into the single applicator 10. However, it is not limited to this configuration. For example, the vector potential coil device 1 and the actuator 3 may be used as separate applicators that are each individually arranged on the human body.

Next, an operation of the treatment device according to the first embodiment will be explained.

The controller 5 controls the vector potential coil drive device 2 and the high frequency power source 4, and causes the vector potential coil device 1 to generate the vector potential, and at the same time, causes the actuator 3 to generate at least one of the ultrashort waves, the microwaves, and the ultrasonic waves, under specified conditions based on the combined therapy of thermotherapy and electrical stimulation.

An alternating current flowing through the VP coil 11 of the vector potential coil device 1 generates an alternating magnetic field along the coil axis. An alternating vector potential is generated in parallel with the alternating current. Therefore, a vector potential is also generated in the affected area. Further, this vector potential generates an electric field so that a voltage is applied to the affected area. In addition, the actuator 3 emits, for instance, the ultrashort waves toward the affected area. The ultrashort waves heat the affected area so that the temperature of the affected area is increased. As a result, the heat and the electrical stimulation according to the above-mentioned combined therapy are applied to the affected area.

As mentioned above, according to the first embodiment, the vector potential coil device 1 generates the vector potential and applies the electrical stimulation to the affected area using the generated vector potential. The actuator 3 generates at least one of the ultrashort waves, the microwaves, and the ultrasonic waves, and heats the affected area with at least one of the ultrashort waves, the microwaves, and the ultrasonic waves. The vector potential coil drive device 2 drives the vector potential coil device 1. The high frequency power source 4 drives the actuator 3. The controller 5 controls the vector potential coil drive device 2 and the high frequency power source 4, and causes the vector potential coil device 1 to generate the vector potential, and at the same time, causes the actuator 3 to generate at least one of the ultrashort waves, the microwaves, and the ultrasonic waves, under specified conditions based on the combined therapy of thermotherapy and electrical stimulation.

Thus, since the need to attach two pads to the skin of the human body for the electrical stimulation is eliminated, the burden on the patient when performing the treatment that combines thermotherapy and electrical stimulation is reduced. Further, since the electrical stimulation is applied in a non-contact manner, energy being applied to the living body by the actuator 3 is less likely to flow back into the electrical stimulation device (here, for instance, the vector potential coil device 1), it is possible to make it less likely that the device will malfunction or be damaged.

### Second Embodiment

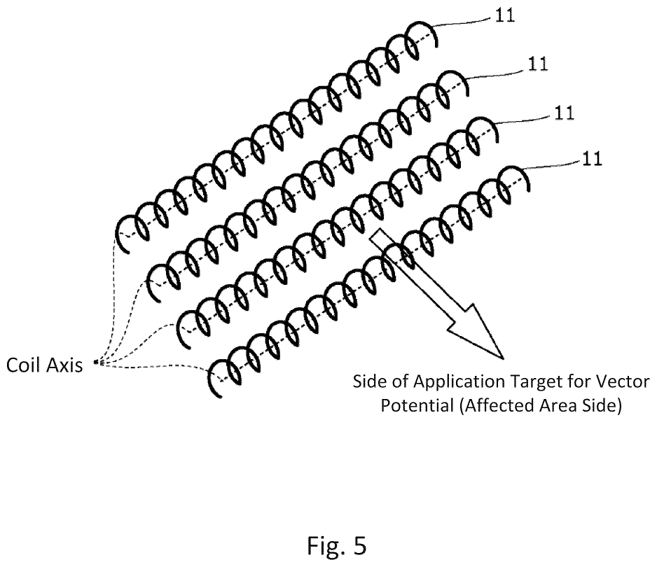

FIG. 5 is a diagram that shows a configuration of a vector potential coil device 1 according to a second embodiment of the present invention. As shown in, for instance, FIG. 5, in the second embodiment, the vector potential coil device 1 has a plurality of VP coils 11. Each VP coil 11 in the second embodiment has a linear (straight) coil axis. Further, the VP coils 11 are a plurality of solenoid coils extending along the coil axes. The plurality of VP coils 11 are arranged along a linear arrangement direction. In other words, the outer shape of the vector potential coil device 1 is in a substantially flat plate shape. The vector potential coil drive device 2 conducts a current through the plurality of VP coils 11. Further, the plurality of VP coils 11 may be electrically connected in series or in parallel to one another. Furthermore, a plurality of vector potential coil drive devices 2 may conduct the current to the plurality of VP coils 11, respectively. In this case, the plurality of vector potential coil drive devices 2 respectively conduct an AC current through the plurality of VP coils 11 under the condition in which the AC currents being conducted through the plurality of VP coils 11 are synchronized.

As mentioned above, by providing the plurality of VP coils 11, the intensity of the vector potential being applied to the application target becomes greater.

Note that the other configurations and operations of the treatment device according to the second embodiment are the same as those explained in any of the other embodiments. Therefore, the explanations of them will be omitted.

### Third Embodiment

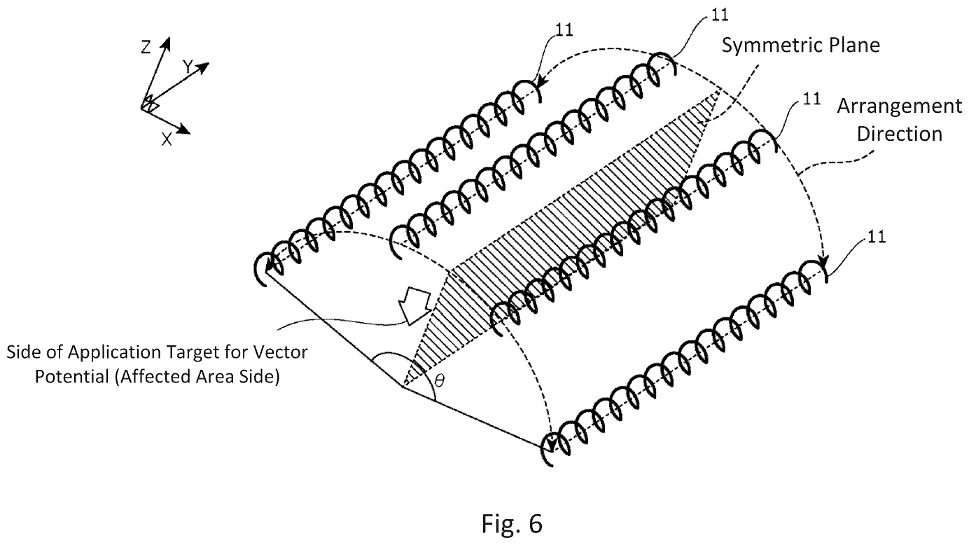

FIG. 6 is a diagram that shows a configuration of a vector potential coil device 1 with respect to a treatment device according to a third embodiment of the present invention. As shown in, for instance, FIG. 6, in the third embodiment, the vector potential coil device 1 has a plurality of VP coils 11. Each VP coil 11 in the third embodiment has a linear coil axis. Further, the VP coils 11 are a plurality of solenoid coils extending along the coil axes. The plurality of VP coils 11 are arranged along a curved (curvature) arrangement direction. The vector potential coil drive device 2 conducts the current through the plurality of VP coils 11. Further, the plurality of VP coils 11 may be electrically connected in series or in parallel to one another. Here, the arrangement direction is a closed curve. Thus, the plurality of VP coils 11 are arranged along the circular-arc-shaped arrangement direction. In particular, the plurality of VP coils 11 are arranged within a range of a predetermined central angle θ (here, at an interval of an equal angle) with respect to a circle that includes the circular arc in the arrangement direction. Since the vector potentials of two of the VP coils 11 are canceled out at the intermediate position between the two VP coils 11, for instance, the central angle θ is set to be any angle less than 180 degrees.

For instance, an affected area of the human body may be arranged within the space in the inner side (direction) of the arranged plurality of VP coils 11. Thus, the vector potential may be applied to that part.

Note that as shown in, for instance, FIG. 6, when the plurality of VP coils 11 having the linear coil axes are arranged plane-symmetrically with respect to a predetermined symmetric plane (a plane perpendicular to the X-axis and parallel to the Z-axis and the Y-axis) along the curved arrangement direction, on an axis that passes through the center of the circle that includes the circular arc in the arrangement direction, and at the same time, is parallel to the coil axes, as a result of a vector synthesis of the vector potentials that are generated by the plurality of VP coils 11, the vector potential is generated in a vertical (perpendicular) direction with respect to the symmetric plane (the X-axis direction in FIG. 6). Therefore, for instance, by combining the VP coil 11 having the curved coil axis shown in FIG. 2 with the plurality of VP coils 11 having the linear coil axes and being arranged plane-symmetrically with respect to the predetermined symmetric plane along the curved arrangement direction, it is possible to generate a vector potential in a desired direction within a two-dimensional plane of the X-axis and Y-axis.

Note that the other configurations and operations of the treatment device according to the third embodiment are the same as those explained in any of the other embodiments. Therefore, the explanations of them will be omitted.

### Fourth Embodiment

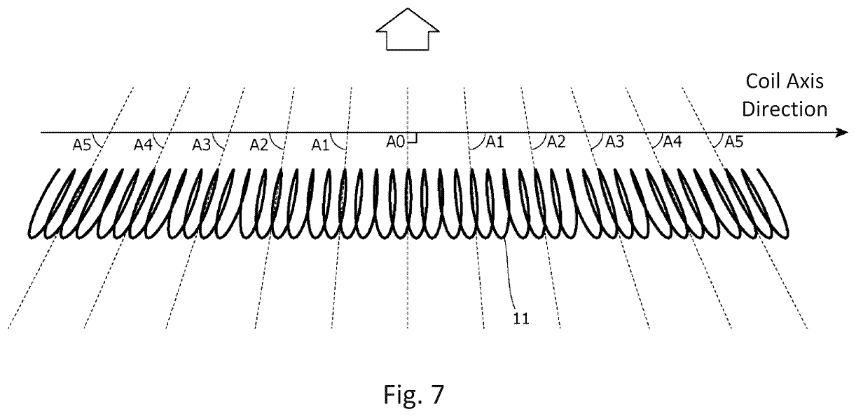

FIG. 7 is a diagram that shows a configuration of a vector potential coil device 1 with respect to a treatment device according to a fourth embodiment of the present invention. In the fourth embodiment, each of the VP coils 11 is wound along (around) a linear coil axis and is wound so that inclination angles in the winding direction gradually change along the direction of the coil axis.

Specifically, as shown in, for instance, FIG. 7, the VP coil 11 is wound along (around) the linear coil axis, and is wound so that the inclination angles (the angles formed by the coil axis direction and the winding direction) A0-A5 in the winding direction gradually change along the direction of the coil axis. More specifically, the inclination angle at the center of the VP coil 11 is 90 degrees. Further, the inclination angle becomes smaller as it moves away from the center (A0>A1>A2>A3>A4>A5). As a result, the above-mentioned vector potential can be applied with satisfactory intensity in the same manner as the case of the curved VP coil 11.

Note that the other configurations and operations of the treatment device according to the fourth embodiment are the same as those explained in any of the other embodiments. Therefore, the explanations of them will be omitted.

### Fifth Embodiment

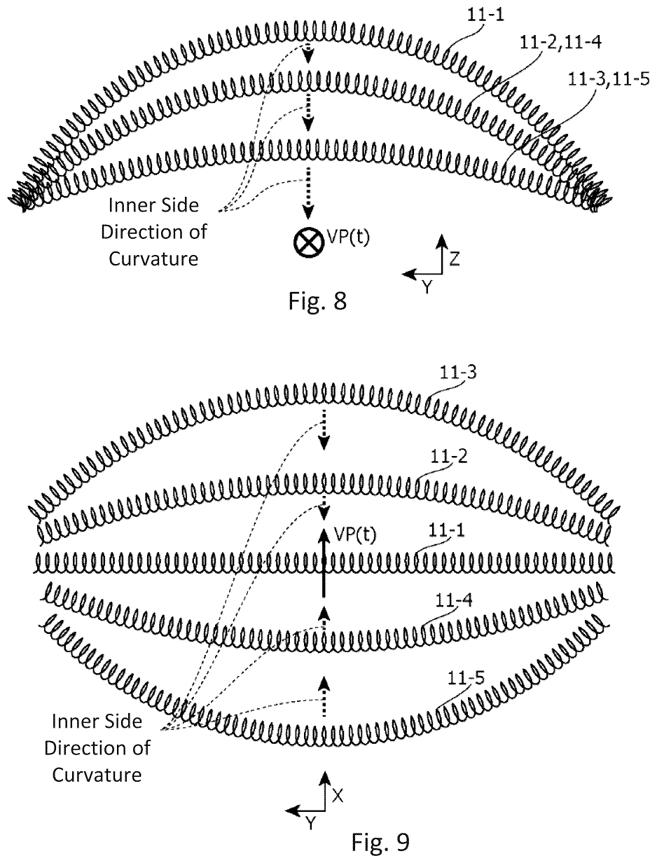

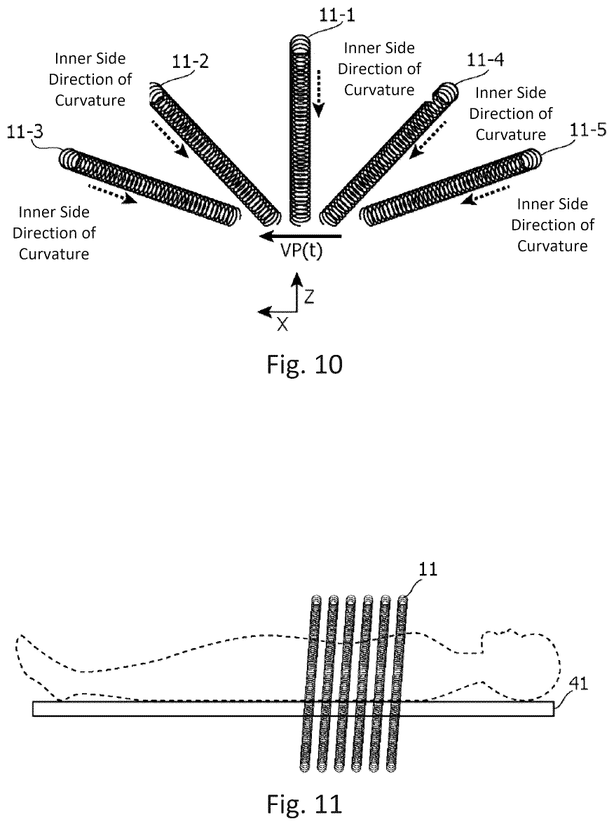

FIG. 8 is a front view that shows an example of a vector potential coil according to a fifth embodiment of the present invention. FIG. 9 is a top view that shows an example of the vector potential coil according to the fifth embodiment of the present invention. FIG. 10 is a side view that shows an example of the vector potential coil according to the fifth embodiment of the present invention.

The vector potential coil device 1 according to the fifth embodiment has a plurality of vector potential coils 11-1-11-5. As shown in, for instance, FIGS. 8-10, the plurality of vector potential coils 11-1-11-5 are respectively wound along (around) a curved coil axis, and are arranged so that the inner sides (directions) of the curvatures of the coil axes (in other words, planes that include the coil axes) cross mutually. For instance, as shown in FIG. 10, the plurality of vector potential coils 11-1-11-5 are arranged so that the planes that include the coil axes of the plurality of vector potential coils 11-1 to 11-5 are parallel to the Y-axis direction, and at the same time, the angle intervals of the inclination angles of these planes with respect to the X-axis direction are substantially the same. In addition, here, the inclination angle of the vector potential coil 11-1 is 90 degrees.

Note that, here, the vector potential coil device 1 has five of the vector potential coils 11-1-11-5. However, the vector potential coil device 1 may have the vector potential coils 11-1-11-M in the same manner as the configuration described above. The number M is either 2-4 coils or 6 or more coils.

For instance, the shape (such as the curvature) and the arrangement of the coil axes are determined so that the coil axes of the plurality of vector potential coils 11-1-11-5 are included in a single partial spherical surface (for instance, a semispherical surface). Further, the application target is arranged at the center of the spherical surface that includes that partial spherical surface (in other words, the center of curvatures of all of the coil axes). Further, the shape (such as the curvature) and the arrangement of the coil axes may be determined so that the coil axes of the plurality of vector potential coils 11-1-11-5 are included in a curved surface (a partial aspherical surface) other than a single partial spherical surface.

Further, the plurality of vector potential coils 11-1-11-5 respectively generate a vector potential according to the AC current in the same manner as the above-mentioned embodiments. The vector potentials by the plurality of vector potential coils 11-1-11-5 are synthesized so that a vector potential VP(t) is obtained. Here, the vector potential coil drive device 2 conducts the AC current through the plurality of vector potential coils 11-1-11-5 so that the amplitude of the synthesized vector potential VP(t) becomes maximum (for instance, in the same phase mutually).

Note that the other configurations and operations of the treatment device according to the fifth embodiment are the same as those explained in any of the other embodiments. Therefore, the explanations of them will be omitted.

As mentioned above, according to the treatment device according to the above-mentioned fifth embodiment, it is possible to concentrate the vector potentials in the inner sides (direction) of the curvatures of the plurality of vector potential coils 11-1-11-5 and apply a high-intensity vector potential to the affected area.

### Sixth Embodiment

FIG. 11 is a side view that shows a treatment device according to a sixth embodiment of the present invention. As shown in FIG. 11, in the sixth embodiment, a VP coil 11 is a solenoid coil being wound around a spiral coil axis. Further, the outer shape of the VP coil 11 is substantially cylindrical.

In addition, a bed 41 for a patient to lie on can be arranged in the hollow portion of the substantially cylindrical VP coil 11. Further, the above-mentioned actuator 3 is incorporated into the bed 41 or an applicator (not shown). As a result, the patient can receive the above-mentioned combined therapy while lying on the bed 41.

Note that the other configurations and operations of the treatment device according to the sixth embodiment are the same as those explained in any of the other embodiments. Therefore, the explanations of them will be omitted.

### Seventh Embodiment

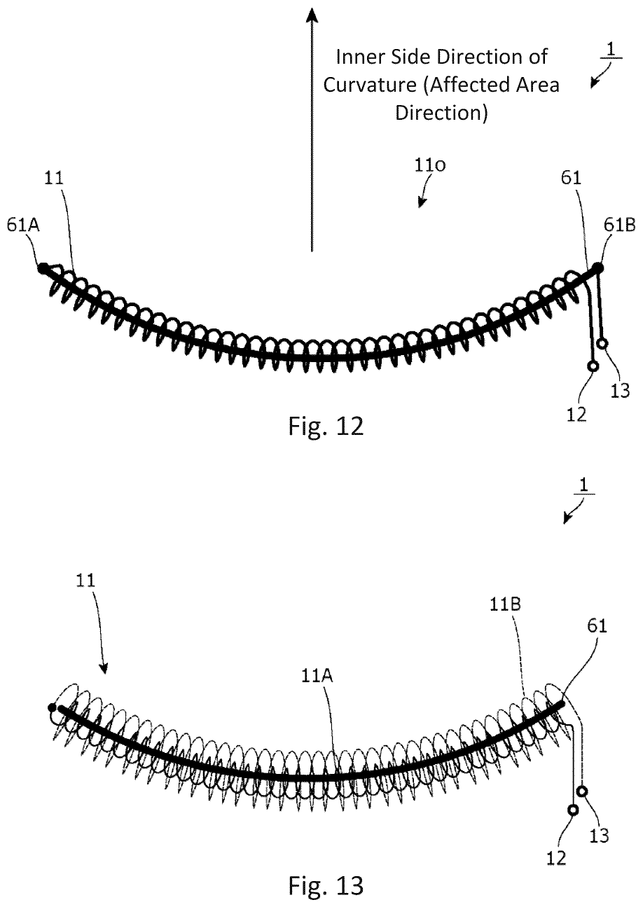

FIG. 12 is a diagram that shows an example of a vector potential coil device 1 according to a seventh embodiment. As shown in, for instance, FIG. 12, the vector potential coil device 1 has a ferromagnetic member 61 that extends along a coil axis that is linear or curved of each of one or a plurality of solenoid coils mentioned above. The ferromagnetic member 61 is formed with a ferromagnetic material. As a result, since the vector potential is enhanced according to an effective magnetic permeability of the ferromagnetic member 61, the intensity of the vector potential becomes greater at the inner side of the curvature (a curvature center in the case of a circular arc shape). Furthermore, an application target to which a vector potential is applied (such as an affected area) may be arranged at the opening 11o of the VP coil 11.

In addition, the ferromagnetic member 61 is formed with a conductive material such as permalloy. Further, since one end of the VP coil 11 and one end (an end 61A) of the ferromagnetic member 61 are electrically connected to each other, the ferromagnetic member 61 forms a path for a current. In addition, the vector potential coil drive device 2 conducts a current through the VP coil 11 by applying a voltage to the other end of the VP coil 11 and the other end of the ferromagnetic member 61. Here, the vector potential coil drive device 2 conducts the current through the VP coil 11 applying the voltage to a terminal 12 being electrically connected to the other end of the VP coil 11 and a terminal 13 being electrically connected to the other end (an end 61B) of the ferromagnetic member 61.

In addition, since the coil axis of the VP coil 11 does not go around once (one revolution) or more, the distance between both ends of the VP coil 11 is large. However, since the ferromagnetic member 61 is used as the current path and two of the terminals 12 and 13 are arranged on either end side of the VP coil 11, the area being encircled by the path through the wiring from the vector potential coil drive device 2 to the VP coil 11 and the ferromagnetic member 61 is relatively narrow. As a result, an unnecessary magnetic field being generated due to the current flowing through the wiring can be suppressed.

Further, the ferromagnetic member 61 shown in FIG. 12 may extend toward an outside of the VP coil 11 (in the outer side (direction) of the curvature) so as to form a closed magnetic path. Furthermore, in that case, a gap may be formed in the closed magnetic path at the outer side of the curvature of the ferromagnetic member 61 and the VP coil 11. Further, the gap may prevent the current from being conducted through an outside part of the curvature of the VP coil 11 in the ferromagnetic member 61.

Note that the other configurations and operations of the treatment device according to the seventh embodiment are the same as those explained in any of the other embodiments. Therefore, the explanations of them will be omitted. In addition, in the seventh embodiment, the ferromagnetic member 61 along the coil axis is added to the VP coil 11 shown in FIG. 12. However, similarly, a ferromagnetic member(s) along the coil axis may be added to the VP coil(s) 11 in the other embodiments.

### Eighth Embodiment

FIG. 13 is a diagram that shows a configuration of a vector potential coil device 1 with respect to a treatment device according to an eighth embodiment of the present invention. In the eighth embodiment, as shown in, for instance, FIG. 13, the VP coil 11 has an inner solenoid coil 11A and an outer solenoid coil 11B that respectively extend along the same curved coil axis and in which coil diameters are different from each other. Further, one coil end of the inner solenoid coil 11A and one coil end of the outer solenoid coil 11B are electrically connected. Each of the inner solenoid coil 11A and the outer solenoid coil 11B functions as one VP coil. Therefore, the VP coil 11 according to the eighth embodiment electrically has a configuration in which two VP coils are connected in series and in the same phase.

The vector potential coil drive device 2 applies a voltage to the other end of the inner solenoid coil 11A and the other end of the outer solenoid coil 11B so that the current is conducted through the VP coil 11. Specifically, the vector potential coil drive device 2 applies the voltage to a terminal 12 being electrically connected to the other end of the inner solenoid coil 11A and a terminal 13 being electrically connected to the other end of the outer solenoid coil 11B so that the current is conducted through the VP coil 11.

Note that the other configurations and operations of the treatment device according to the eighth embodiment are the same as those explained in any of the other embodiments. Therefore, the explanations of them will be omitted.

### Ninth Embodiment

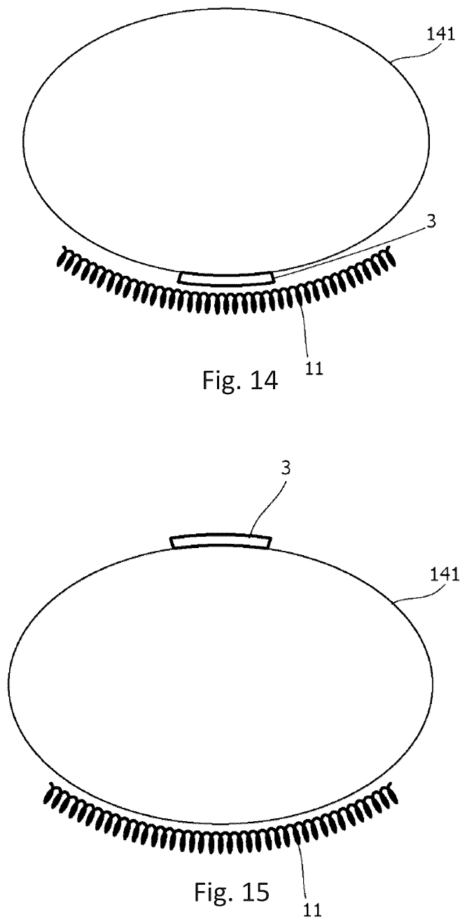

FIG. 14 is a diagram that explains an arrangement of a vector potential coil device 1 (a VP coil 11) and an actuator 3 relative to a human body according to a ninth embodiment. In the ninth embodiment, in FIG. 4, the actuator 3 generates ultrashort waves using a coil 3a having a planar coil shape (see FIG. 23).

As mentioned above, the vector potential coil device 1 (the VP coil 11) can apply the vector potential and its fluctuations (changes) to the affected area regardless of a material that exists between the affected area and the VP coil 11. On the other hand, since the ultrashort wave energy being irradiated from the coil 3a to the affected area becomes weaker as the distance between the affected area and the coil 3a increases, it is preferred that the coil 3a is arranged in contact with or close to contact with the affected area or the skin of the human body when it is used.

In the ninth embodiment, as shown in, for instance, FIG. 14, both the vector potential coil device 1 (the VP coil 11) and the actuator 3 (the coil 3a) are arranged on either the front side or the back side of a human body 141. Further, the actuator 3 (the coil 3a) is arranged between the vector potential coil device 1 (the VP coil 11) and the human body 141. Note that both the vector potential coil device 1 (the VP coil 11) and the actuator 3 (the coil 3a) may be arranged on the right side or the left side of the human body 141. Furthermore, the applicator 10 is configured accordingly so that the vector potential coil device 1 (the VP coil 11) and the actuator 3 (the coil 3a) are arranged in this manner.

Note that the other configurations and operations of the treatment device according to the ninth embodiment are the same as those explained in any of the other embodiments. Therefore, the explanations of them will be omitted.

Thus, in the ninth embodiment, the vector potential coil device 1 (the VP coil 11) and the actuator 3 (the coil 3a) are configured to overlap and be arranged only on either the front side or the back side of the human body. Therefore, there is no need to arrange the treatment device on the other side (the front side or the back side). Thus, the patient can receive the treatment using the treatment device, for instance, while lying on their back on an examination table or a bed so that the burden on the patient can be reduced.

### Tenth Embodiment

FIG. 15 is a diagram that explains an arrangement of a vector potential coil device 1 (a VP coil 11) and an actuator 3 relative to a human body according to a tenth embodiment. In the tenth embodiment, the actuator 3 generates ultrashort waves using a coil 3a having a planar coil shape.

In the tenth embodiment, as shown in, for instance, FIG. 15, the vector potential coil device 1 (the VP coil 11) is arranged on one side of the front side and the back side of a human body 141. Further, the actuator 3 (the coil 3a) is arranged on the other side of the front side and the back side. Note that the vector potential coil device 1 (the VP coil 11) may be arranged on one side of the right side and the left side of the human body 141. Further, the actuator 3 (the coil 3a) may be arranged on the other side of the right side and the left side. In addition, the vector potential coil device 1 (the VP coil 11) may be arranged on one side of the front, back, right, and left sides of the human body 141. Further, the actuator 3 (the coil 3a) may be arranged on another side of the remaining sides. In other words, in the tenth embodiment, the vector potential coil 11 and the actuator 3 are arranged on opposite sides each other with the human body 141 in the center so that the human body 141 is sandwiched between the vector potential coil 11 and the actuator 3. Furthermore, the applicator 10 is configured accordingly so that the vector potential coil device 1 (the VP coil 11) and the actuator 3 (the coil 3a) are arranged in this manner.

Note that the other configurations and operations of the treatment device according to the tenth embodiment are the same as those explained in any of the other embodiments. Therefore, the explanations of them will be omitted.

As mentioned above, in the tenth embodiment, the vector potential coil device 1 (the VP coil 11) and the actuator 3 (the coil 3a) are arranged so that the human body 141 is sandwiched between the vector potential coil 11 and the actuator 3. As a result, both the vector potential coil 11 and the actuator 3 are arranged in the state of being closer to the human body 141. Further, in the tenth embodiment, since the vector potential coil device 1 (the VP coil 11) and the actuator 3 (the coil 3a) are arranged at a distance via the human body 141, the vector potential coil drive device 2 and the high frequency power source 4 are less likely to interfere with or interact with each other.

Note that the driving frequency of the VP coil 11 is from several tens to several hundreds of kHz. The driving frequency of the coil 3a in the case of the ultrashort waves is substantially 27 MHz. Although the two frequencies are significantly different, depending on the power being input to the VP coil 11 and the coil 3a, there is a possibility that the interaction may occur due to the frequencies of the two. Therefore, as in the tenth embodiment, by arranging the two components at a distance each other, such interaction is less likely to occur, and driving with a larger power becomes possible. As mentioned above, it is preferred to arrange the vector potential coil drive device 2 and the high frequency power source 4 at a distance each other from the viewpoint of the stable operation of both the vector potential coil drive device 2 and the high frequency power source 4. Note that, 27 MHz is used as the frequency of the ultrashort waves because this is based on the frequency based on Japanese laws regarding the ultrashort wave therapeutic devices. When used overseas, the frequency band based on the laws of that country regarding therapeutic devices should be used as the ultrashort waves. Thus, the ultrashort wave is not limited to 27 MHz or the above-mentioned 30-300 MHz as an example of a general frequency of an ultrashort wave.

### Eleventh Embodiment

A treatment device according to an eleventh embodiment is the same as that according to the ninth embodiment, except that an actuator 3 is a capacitor type.

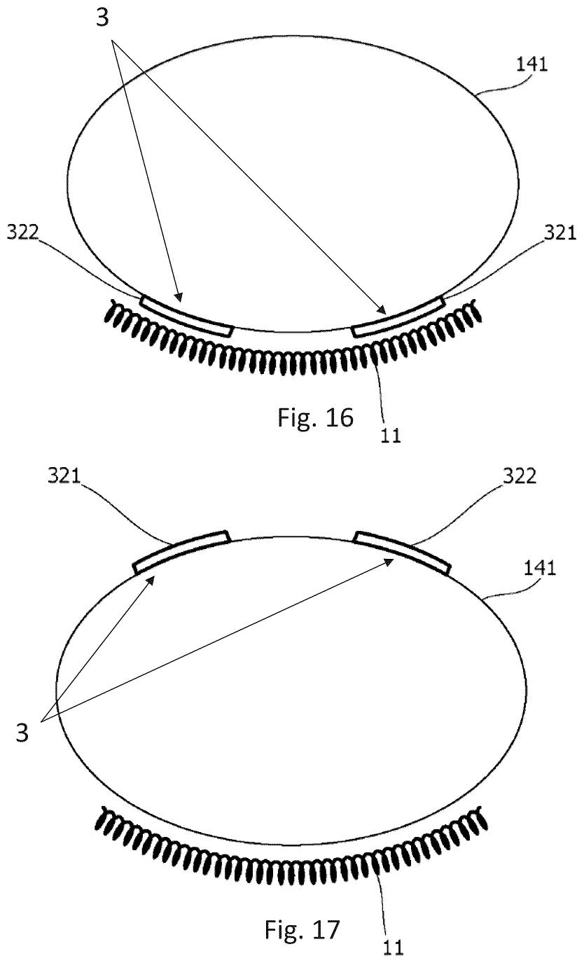

FIG. 16 is a diagram that explains an arrangement of a vector potential coil device 1 (the VP coil 11) and an actuator 3 relative to a human body according to the eleventh embodiment. In the eleventh embodiment, the actuator 3 is the capacitor type and generates such as the above-mentioned ultrashort waves by using at least two flat plate-shaped electrodes 321 and 322.

In the eleventh embodiment, as shown in, for instance, FIG. 16, both the vector potential coil device 1 (the VP coil 11) and the actuator 3 (the electrodes 321 and 322) are arranged on one side of the front side and the back side of the human body 141. Further, the actuator 3 (the electrodes 321 and 322) is arranged between the vector potential coil device 1 (the VP coil 11) and the human body 141. Note that both the vector potential coil device 1 (the VP coil 11) and the actuator 3 (the electrodes 321 and 322) may be arranged on the right side or the left side of the human body 141. Furthermore, an applicator 10 is configured accordingly so that the vector potential coil device 1 (the VP coil 11) and the actuator 3 (the electrodes 321 and 322) are arranged in this manner.

Note that the other configurations and operations of the treatment device according to the eleventh embodiment are the same as those explained in the ninth embodiment. Therefore, the explanations of them will be omitted.

### Twelfth Embodiment

A treatment device according to a twelfth embodiment is the same as that according to the tenth embodiment, except that the actuator 3 is a capacitor type.

FIG. 17 is a diagram that explains an arrangement of a vector potential coil device 1 (the VP coil 11) and an actuator 3 relative to a human body according to the twelfth embodiment. In the twelfth embodiment, the actuator 3 is a capacitor type and generates such as the above-mentioned ultrashort waves by using a pair of flat plate-shaped electrodes 321 and 322.

In the twelfth embodiment, as shown in, for instance, FIG. 17, the vector potential coil device 1 (the VP coil 11) is arranged on one side of the front side and the back side of the human body 141. Further, the actuator 3 (the electrodes 321 and 322) is arranged on the other side of the front side and the back side. Note that the vector potential coil device 1 (the VP coil 11) may be arranged on one side of the right side and the left side of the human body 141. Further, the actuator 3 (the electrodes 321 and 322) may be arranged on the other side of the right side and the left side. In addition, the vector potential coil device 1 (the VP coil 11) may be arranged on one side of the front, back, right, and left sides of the human body 141. Further, the actuator 3 (the electrodes 321 and 322) may be arranged on one of the remaining sides. In other words, in the twelfth embodiment, the vector potential coil 11 and the actuator 3 are arranged on opposite sides each other with the human body 141 as the center so that the human body 141 is sandwiched between the vector potential coil 11 and the actuator 3. Furthermore, an applicator 10 is configured accordingly so that the vector potential coil device 1 (the VP coil 11) and the actuator 3 (the electrodes 321 and 322) are arranged in this manner.

Note that the other configurations and operations of the treatment device according to the twelfth embodiment are the same as those explained in the tenth embodiment. Therefore, the explanations of them will be omitted.

### Thirteenth Embodiment

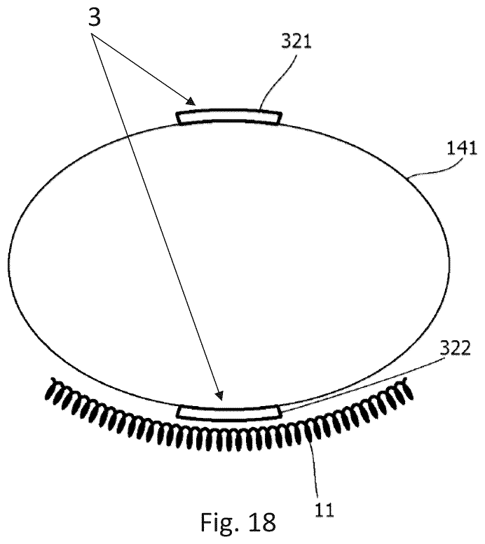

FIG. 18 is a diagram that explains an arrangement of a vector potential coil device 1 (the VP coil 11) and an actuator 3 relative to a human body according to a thirteenth embodiment. In the thirteenth embodiment, the actuator 3 is a capacitor type and generates such as the above-mentioned ultrashort waves by using a pair of flat plate-shaped electrodes 321 and 322. Note that the arrangement location of the pair of electrodes 321 and 322 according to the thirteenth embodiment are different from the arrangement location of the pair of electrodes 321 and 322 according to the eleventh and twelfth embodiments.

In the thirteenth embodiment, as shown in, for instance, FIG. 18, the vector potential coil device 1 (the VP coil 11) is arranged on one side of the front side and the back side of the human body 141. Further, the electrodes 321 and 322 of the actuator 3 are respectively arranged on the front side and the back side of the human body 141. In other words, the vector potential coil device 1 (the VP coil 11) and one of the electrodes 321 and 322 of the actuator 3 are arranged on one side of the front side and the back side of a human body 141. Further, the other of the electrodes 321 and 322 of the actuator 3 is arranged on the other side of the front side and the back side of the human body 141. Further, one of the electrodes 321 and 322 may be arranged on one side of the right side and the left side of the human body 141. Further, the other of the electrodes 321 and 322 may be arranged on the other side of the right side and the left side of the human body 141. Furthermore, the vector potential coil device 1 may be arranged on either the right side or the left side of the human body 141. Furthermore, an applicator 10 is configured accordingly so that the vector potential coil device 1 (the VP coil 11) and the actuator 3 (the electrodes 321 and 322) are arranged in this manner.

Note that the other configurations and operations of the treatment device according to the thirteenth embodiment are the same as those explained in any of the other embodiments. Therefore, the explanations of them will be omitted.

### Fourteenth Embodiment

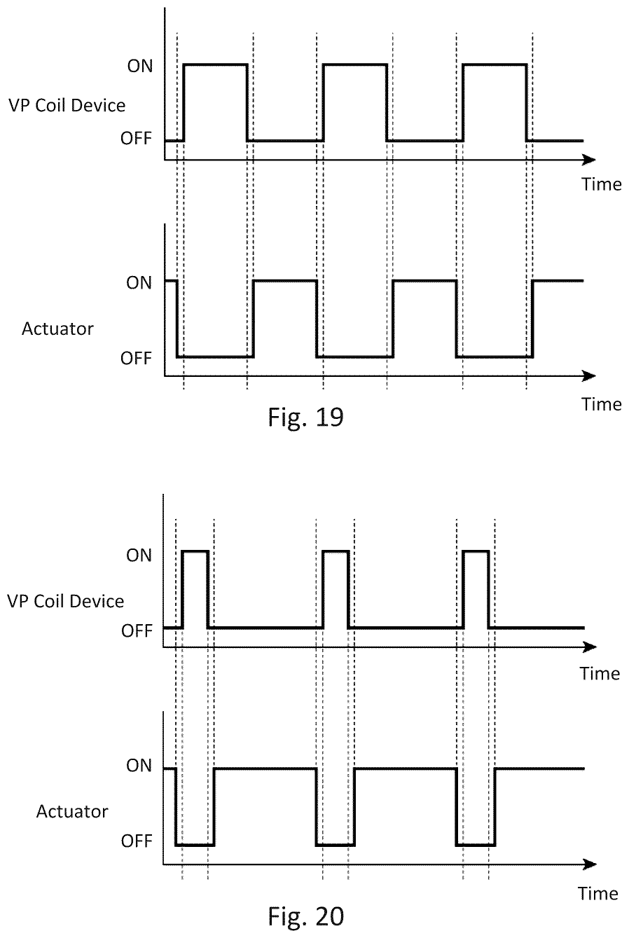

FIGS. 19-21 are diagrams that show examples of drive waveforms of a vector potential coil device 1 and an actuator 3 with respect to a treatment device according to a fourteenth embodiment.

In the fourteenth embodiment, as shown in, for instance, FIGS. 19-2 , the controller 5 performs a burst control on the vector potential coil drive device 2 and the high frequency power source 4 so that the output of the vector potential from the vector potential coil device 1 and the output of such as the ultrashort waves from the actuator 3 are not performed simultaneously, and at the same time, so that each output alternates between an on state and an off state. Note that, even when the outputs are alternately turned on and off in this way, the vector potential is applied in the state in which the temperature of the affected area is maintained.

Further, in the above-mentioned burst control, the controller 5 may be configured to (a) turn on and off operations of the vector potential coil drive device 2 and the high frequency power source 4, or (b) provide switching means (for instance, switching elements such as transistors or relays, or other electric circuits) (not shown) between the vector potential coil drive device 2 and the high frequency power source 4 and the vector potential coil device 1 and the actuator 3, respectively, keep the operations of the vector potential coil drive device 2 and the high frequency power source 4 are continuously turned on, and turn on and off the outputs from the vector potential coil drive device 2 and the high frequency power source 4 to the vector potential coil device 1 and the actuator 3, respectively, by using the switching means.

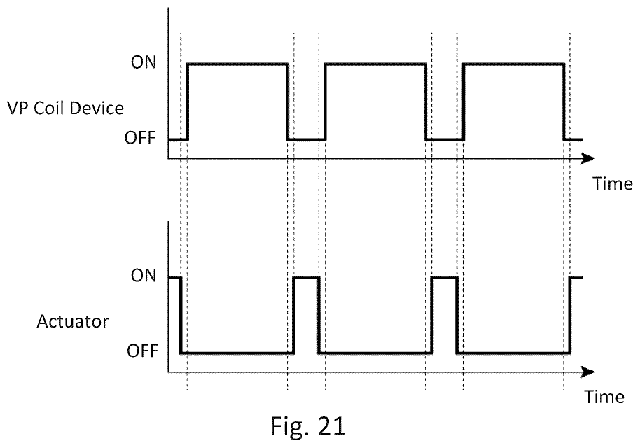

Further, a duty ratio of the output of the vector potential and a duty ratio of the output of such as the ultrashort waves may each be constant or may be adjusted appropriately. FIG. 19 shows a case in which the duty ratios of both are substantially the same. FIG. 20 shows a case in which the duty ratio of the output of the vector potential is lower than the duty ratio of the output of such as the ultrashort waves. FIG. 21 shows a case in which the duty ratio of the output of the vector potential is higher than the duty ratio of the output of such as the ultrashort waves.

For instance, during a predetermined period at the start of treatment, since the temperature of the affected area is low, as shown in, for instance, FIG. 20, the controller 5 may prioritize the output of such as the ultrashort waves by making the output-on period of such as the ultrashort waves longer than the output-on period of the vector potential.

Further, thereafter, the controller 5 may gradually or in stages shorten the output-on period of such as the ultrashort waves, and gradually or in stages lengthen the output-on period of the vector potential, as the treatment time elapses. Alternatively, immediately after that, the duty ratios of both may transition to a state in which they are substantially the same, as shown in FIG. 19.

Furthermore, after the sufficient heat from such as the ultrashort waves has been applied to the affected area, as shown in, for instance, FIG. 21 (opposite to FIG. 20), the controller 5 may control the vector potential coil drive device 2 and the high frequency power source 4 so as to shorten the output-on period of such as the ultrashort waves and lengthen the output-on period of the vector potential.

Furthermore, the controller 5 may perform a control to gradually lengthen the output-on period of the vector potential and/or perform a control to shorten the output-on period of such as the ultrashort waves, continuously over time since the beginning of the treatment. In addition, the controller 5 may be configured to only output such as the ultrashort waves without outputting the vector potentials for a predetermined time since the beginning of the treatment (the time until a predetermined temperature rise occurs in the affected area).

As a result, because the timing of the peak instantaneous power consumptions of the vector potential coil drive device 2 and the high frequency power source 4 do not overlap, the maximum value of the instantaneous power consumption of the treatment device is suppressed. Furthermore, when one of the vector potential coil drive device 2 and the high frequency power source 4 is driving one of the vector potential coil device 1 and the actuator 3, the other of the vector potential coil drive device 2 and the high frequency power source 4 is not driving the other of the vector potential coil device 1 and the actuator 3. Therefore, problems such as circuit failure or malfunction due to the interference or the interaction between the vector potential coil drive device 2 and the high frequency power source 4 are suppressed. Further, at the same time, noise countermeasures between the two are reduced. As a result, the miniaturization of the device and the cost reduction can be realized.

Note that the other configurations and operations of the treatment device according to the fourteenth embodiment are the same as those explained in any of the other embodiments. Therefore, the explanations of them will be omitted.

### Fifteenth Embodiment

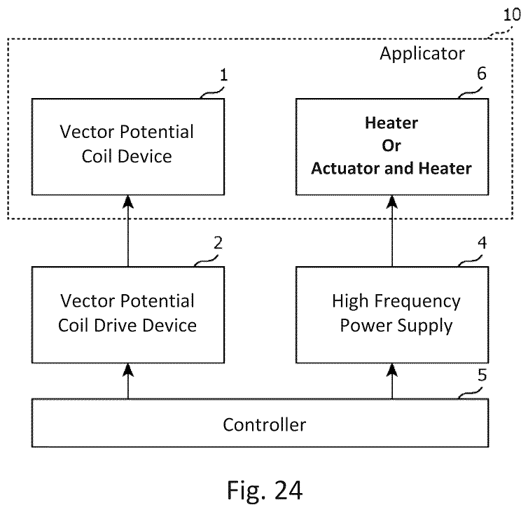

In a fifteenth embodiment, as shown in FIG. 24, a heater 6 incorporating a heating element is used as the heating means instead of the above-mentioned actuator 3, or together with the above-mentioned actuator 3. For instance, a planar (sheet) heater in which a nickel resistor (a heating element) is printed on a substrate such as silicon is used as this heater 6.

Even when the heater 6 is used as the heating means, the controller 5 may perform the burst control as mentioned above. In this case, the controller 5 controls the heater 6 to turn on and off such that turning on the heater 6 during the period in which the output of the vector potential is turned off and turning off the heater 6 during the period in which the output of the vector potential is turned on.

Further, in case of using the heater 6 together with the above-mentioned actuator 3, when the coil 3a having a planar coil shape (see FIG. 23) is used as the actuator 3, the actuator 3 (the coil 3a) and the heater 6 may be respectively arranged opposite each other with the human body 141 at the center, as in the case of the electrodes 321 and 322 according to the thirteenth embodiment.

Furthermore, in case of using the heater 6 together with the above-mentioned actuator 3, when the actuator 3 of a capacitor type is used, the heater 6 may be arranged between the electrodes 321 and 322 according to the eleventh and twelves embodiments, or may be arranged on the opposite side of the electrodes 321 and 322 with respect to the human body 141.

Note that the other configurations and operations of the treatment device according to the fifteenth embodiment are the same as those explained in any of the other embodiments. Therefore, the explanations of them will be omitted.

Note that various changes and modifications to the embodiments described above will be apparent to one having ordinally skill in the art. Such the changes and modifications may be made without departing from the spirit and scope of the subject matter and without diminishing the intended advantages. That is, it is intended that such the changes and modifications are included within the scope of the claims.

For instance, in the above-mentioned first embodiment, the coil axis of the VP coil 11 may have a spiral shape along a plane or a spherical surface.

Further, in the above embodiments, when a plurality of VP coils 11 are used, it is preferred to connect these VP coils 11 in series, since this configuration reduces the current capacities of the amplifier circuit 22 and the matching circuit 23. Due to the reduction of the current capacities, the sizes of the amplifier circuit 22 and the matching circuit 23 are reduced. Further, at the same time, the heat generation is reduced. As a result, the cost of the device is reduced, and at the same time, the breakdowns of the device become less likely.

In addition, the applicator 10 according to the ninth-thirteenth embodiments may be a belt-shaped applicator 10 in which the VP coil 11 and the electrodes 321 and 322 or the coil 3a (the actuator 3) are arranged in predetermined positions. Further, the belt-shaped applicator 10 may be capable of being wrapped around the human body 141. In this case, in the belt-shaped applicator 10, the VP coil 11 and the electrodes 321 and 322 or the coil 3a (the actuator 3) are fixed so that when wrapped around the human body 141, the VP coil 11 and the electrodes 321 and 322 or the coil 3a (the actuator 3) are arranged as mentioned above. For example, FIG. 23 shows a perspective view of an applicator 10 that is belt-shaped according to a modified embodiment. The belt-shaped applicator 10 includes a belt-shaped vector potential coil device 1 and the coil 3a (the actuator 3). The belt-shaped vector potential coil device 1 is intended to be wrapped around a trunk of the human body, arms, or legs (limbs). The belt-shaped vector potential coil device 1 has a belt-shaped housing (belt-shaped base) that is made of a flexible material in which the VP coil 11 is (the VP coils are) contained. As the actuator 3, for example, the planar coil 3a that is in a planar shape is preferably used. The planar coil 3a is located at a position where the planar coil 3a is contacted to or directly adjacent to the human body 141 when the belt-shaped applicator 10 is wrapped around the human body 141. Alternatively, the planar coil 3a is arranged within the belt-shaped vector potential coil device 1 (belt-shaped housing). The belt-shaped vector potential coil device 1 is wrapped around an affected part, such as an abdominal region or an arm, to be fixed to the body of the patient. Therefore, the hyperthermia treatment (thermotherapy) and electrical stimulation can be stably applied at the affected part of the patient.

Note that the VP coil 11 being used in the ninth-thirteenth embodiments is the VP coil 11 shown in FIG. 2. However, it is not limited to this configuration. Instead, the VP coil in the other embodiments (FIGS. 5-10, FIG. 12, and FIG. 13) may be used. In addition, in the sixth embodiment, since the human body 141 is arranged in the hollow portion of the VP coil 11, it is preferred that the actuator 3 is arranged between the VP coil 11 and the human body so as to be as close as possible to the human body 141.

Note that, in FIG. 2, FIGS. 5-11, and FIGS. 14-18, the illustration of the ferromagnetic member 61 is omitted from these drawings.

### INDUSTRIAL APPLICABILITY

The present invention can be applicable to, for instance, a treatment device for a combined therapy of thermotherapy and electrical stimulation.

What is claimed is:

1. A treatment device for a combined therapy of thermotherapy and electrical stimulation, the treatment device comprising:
a vector potential coil device that generates a vector potential and applies electrical stimulation to an affected area using the generated vector potential;
a heating means that heats the affected area;
a vector potential coil drive device that drives the vector potential coil device;
a high frequency power supply that drives the heating means; and
a controller that controls the vector potential coil drive device and the high frequency power supply, causes the vector potential coil device to generate the vector potential, and causes the heating means to heat the affected area under a specified condition based on the combined therapy of thermotherapy and electrical stimulation.

2. The treatment device according to claim 1,
wherein the heating means includes an actuator that generates at least one of an ultrashort wave, a microwave, and an ultrasonic wave and heats the affected area with at least one of the ultrashort wave, the microwave, and the ultrasonic wave,
the high frequency power supply drives the actuator, and
the controller controls the vector potential coil drive device and the high frequency power supply to cause the vector potential coil device to generate the vector potential and cause the actuator to generate at least one of the ultrashort wave, the microwave, and the ultrasonic wave under a predetermined condition based on the combined therapy of hyperthermia and electrical stimulation.

3. The treatment device according to claim 1,
wherein the vector potential coil device includes one or a plurality of solenoid coils arranged along a predetermined planar shape or a predetermined curved shape, and
the vector potential is generated by the one or the plurality of solenoid coils.

4. The treatment device according to claim 3,
wherein the vector potential coil device includes a ferromagnetic member that extends along a linear coil axis or a curved coil axis of each of the one or the plurality of solenoid coils.

5. The treatment device according to claim 2, further comprising: a single applicator,
wherein the vector potential coil device and the actuator are incorporated into the single applicator.

6. The treatment device according to claim 5,
wherein the applicator is not directly attached to a skin of a human body, and
the vector potential coil device and the actuator are disposed at a position where the vector potential and at least one of the ultrashort wave, the microwave, and the ultrasonic wave are applied to the affected area.

7. The treatment device according to claim 2,
wherein the controller controls the vector potential coil drive device and the high frequency power supply such that:
output of the vector potential from the vector potential coil device and output of at least one of the ultrashort wave, microwave, and ultrasonic wave from the actuator are not performed simultaneously; and
each of the outputs is repeatedly alternately on and off.

8. The treatment device according to claim 7,
wherein the controller adjusts a duty ratio of the output of the vector potential and a duty ratio of the output of at least one of the ultrashort wave, the microwave, and the ultrasonic wave according to an elapsed time from a start of a treatment.

9. The treatment device according to claim 1,
wherein the heating means is disposed between the vector potential coil device and a human body.

10. The treatment device according to claim 1,
wherein the heating means is arranged on an opposite side of the vector potential coil device with a human body at a center.

11. The treatment device according to claim 1,
wherein the heating means includes a heater that generates heat to heat the affected area.

12. The treatment device according to claim 1,
wherein the combined therapy activates a normalization mechanism in biological tissues in the affected area via heat shock proteins or ubiquitinated proteins.

13. The treatment device according to claim 2,
wherein the combined therapy activates a normalization mechanism in biological tissues in the affected area via heat shock proteins or ubiquitinated proteins.

14. The treatment device according to claim 3,
wherein the combined therapy activates a normalization mechanism in biological tissues in the affected area via heat shock proteins or ubiquitinated proteins.

15. The treatment device according to claim 4,
wherein the combined therapy activates a normalization mechanism in biological tissues in the affected area via heat shock proteins or ubiquitinated proteins.

16. The treatment device according to claim 5,
wherein the combined therapy activates a normalization mechanism in biological tissues in the affected area via heat shock proteins or ubiquitinated proteins.

17. The treatment device according to claim 6,
wherein the combined therapy activates a normalization mechanism in biological tissues in the affected area via heat shock proteins or ubiquitinated proteins.

18. The treatment device according to claim 7,
wherein the combined therapy activates a normalization mechanism in biological tissues in the affected area via heat shock proteins or ubiquitinated proteins.

19. The treatment device according to claim 8,
wherein the combined therapy activates a normalization mechanism in biological tissues in the affected area via heat shock proteins or ubiquitinated proteins.

20. The treatment device according to claim 9,
wherein the combined therapy activates a normalization mechanism in biological tissues in the affected area via heat shock proteins or ubiquitinated proteins.
